(19) United States
(12) Patent Application Publication
(10) Pub. No.: US 2007/0203548 A1
(43) Pub. Date: Aug. 30, 2007

(54) System And Device Implantable In Tissue Of A Living Being For Recording And Influencing Electrical Bio-Activity

(76) Inventors: Klaus Richard Pawelzik, Bremen (DE); David Rotermund, Delmenhorst (DE)

Correspondence Address: EDWARD W. GOEBEL, JR.;MACDONALD, ILLIG, JONES & BRITTON LLP, 100 STATE STREET, SUITE 700, ERIE, PA, 16507-1498, US

(21) Appl. No.: 10/594,051

(22) PCT Filed: Mar. 22, 2005
(86) PCT No.: PCT/DE05/00521

§ 371 Date: Nov. 13, 2006

(30) Foreign Application Priority Data
Mar. 25, 2004 (DE) 10 2004 014 694.2

(51) Int. Cl. A61N 1/18; A61N 1/00

(52) U.S. Cl. 607/61; 600/554

(57) ABSTRACT

Devices are implantable in tissue of living beings for detecting and influencing electrical bioactivity.

### BACKGROUND

The present invention is a device implantable in tissue of living beings for detecting and/or influencing electrical bioactivity.

Implantable devices for detecting and/or influencing electrical bioactivity include those disclosed in U.S. Patent Application Publication No. 2003/0114769 A1, and International Application WO 00/13585. However, such devices are very large and are therefore unsuited for use in certain types of biological systems, such as in, for example, central nervous systems.

### SUMMARY

A device for implantation in tissue of a living being allows for detecting electrical bioactivity of the tissue. Two measuring electrodes are positioned to detect a voltage difference representing bioactivity of the tissue. A wireless transmitter transmits information outside the tissue, the information relating to the bioactivity as represented by the voltage difference detected by the two measuring electrodes. A wireless energy receiver for receiving energy from outside the tissue supplies the transmitter with electrical energy, the transmitter and the energy receiver operating in parallel in time. A voltage sensitive switch is connected between the two measuring electrodes and the transmitter. The voltage sensitive switch is positioned for switching the transmitter such that information relating to changes in electrical bioactivity can be coded in analog fashion in the form of a change of at least one transmission property of the transmitter, and information relating to the identity of the transmitter can be coded in analog fashion in the form of at least one transmission property of the transmitter.

The invention also allows for a device to be implanted in tissue for influencing electrical bioactivity. In such an arrangement, two electrodes are positioned to apply an electric voltage in the tissue to influence bioactivity. An energy receiver for receiving energy from outside the tissue supplies the two electrodes with electrical energy. A control information receiver is positioned to receive wireless control information signals from outside the tissue, the energy receiver and control information receiver operating in parallel in time. A voltage-sensitive switch is connected between the control information receiver and the two electrodes, the voltage-sensitive switch being positioned for switching a flow of electric current from the energy receiver to the electrodes under the control of the control information receiver. The identity of the control information receiver and the magnitude of the influence on the electrical bioactivity are coded in analog fashion by at least one of the frequency and/or amplitude of the control information signals.

The invention allows for the miniaturization of devices for exchanging signals between biological systems and units located outside thereof such as, for example, measuring, monitoring and control units, so-called stimulators or effectors.

Where the invention is employed to detect bioactivity, an example of coding in analog fashion of information relating to changes in bioactivity and information relating to the identity of the transmitter includes coding in analog fashion the time profile of the voltage difference to be coded and/or imaged into a change in, for example, the transmit amplitude, transmit wavelength, transmit frequency, or alternatively, in the shape and level of individual pulses.

Where the invention is employed to influence electrical bioactivity, the identity of the control information receiver and magnitude of the influence on the electrical bioactivity being coded in analog fashion can be manipulated by manipulating the frequency and/or amplitude of the control information signals.

It will be appreciated that the tissue being detected for bioactivity or having its bioactivity influenced can be a tissue inside or outside a living animal or human being. In particular, the invention is useful for implantation in the brain, heart or in the musculature such that the invention can be used for medical diagnostics, neurophysiology and in the control of prostheses.

It will be further appreciated that the electrical bioactivity is intended to include the membrane voltage or the temporal change therein of cells, such as nerve cells.

In some embodiments, the invention can be configured for detecting electrical bioactivity where the transmission properties include the transmit amplitude and/or the transmit frequency.

Where the invention is employed to detect electrical bioactivity, the switch can be configured to switch the transmitter on or off when the detected voltage difference overshoots or undershoots a voltage threshold value which can be fixed in advance. The presence of an action potential, i.e. a sudden change in a membrane voltage such as for nerve cells inside and outside the brain, can thereby be detected and passed on. The switch then acts like a 1-bit switch.

In some embodiments, the transmitter can comprise a closed resonant circuit, particularly where microwaves and radio waves are utilized by the implemented invention. Alternatively, where IR, UV and visible light are utilized, the transmitter can comprise a photodiode. In some embodiments, the transmitter can comprise an LED. The transmitter can also comprise a quantum well structure, for example, where a quantum laser is involved. In some embodiments, the transmitter can comprise a quantum line structure.

Some embodiments can include at least two transmitters that can be distinguished on the basis of having different analog transmission properties, e.g. transmit amplitude and/or transmit frequency. It is thereby possible, firstly, to achieve an even higher density of the devices in a tissue, and also to achieve a unique identification of the transmitters without a large outlay on components and signal processing.

Where the invention is employed to influence electrical bioactivity, such as where the invention is used as a microeffector or microstimulator, the switch can be driven by the control information receiver such that a voltage pulse is generated between the electrodes. If the voltage pulse is sufficiently strong and short, surrounding cells can be stimulated to bioactivity. Alternatively, in place of a voltage pulse, a voltage profile controlled from outside the tissue can be output or induced in the surrounding tissue.

In some embodiments, the control information receiver can comprise a closed resonant circuit, especially where microwaves and radio waves are used. Alternatively, if IR, UV and/or visible light are used, the control information receiver can comprise a photodiode.

Some embodiments include two control information receivers that can be addressed separately on the basis of different analog reception properties (amplitude and/or frequency). Such embodiments allow for a higher density of control of and separate driving of the control information receivers.

In some embodiments, the energy receiver can comprise a closed resonant circuit, especially where microwaves and radio waves are used. Alternatively, the energy receiver can comprise a photodiode, especially where IR, UV and/or visible light are/is used. In further embodiments, the energy receiver can comprise a piezocrystal if sound waves are used.

In one particularly simple embodiment, the voltage-sensitive switch can comprise a voltage-sensitive resistor. Alternatively, the voltage-sensitive switch can comprise a chain of open field effect transistors. In other embodiments, the voltage-sensitive switch can comprise an electrooptic switch. Where an electrooptic switch is used, the electrooptic switch may comprise an LED and a photodiode.

In some embodiments of the invention, one or more component devices can be advantageously included in an integrated circuit (IC).

With the exception of contact points of the measuring electrodes and/or electrodes, the invention is typically enclosed within an electrically insulating material, such as varnish. Such enclosure can minimize stimulation of the tissue, especially brain tissue.

In some embodiments, measuring electrodes and/or electrodes can be configured as or integrated into a spur to further minimize tissue stimulations. In other embodiments, the measuring electrodes and/or electrodes can be configured as or integrated into a capacitor or a spur with a capacitor.

It is contemplated that a system for detecting and/or influencing electrical bioactivity comprising at least two devices implanted in a tissue and/or living being is within the intended scope of the invention. In particular, it can be provided that at least one energy transmission device and at least one bioactivity detection device and/or at least one bioactivity influencing device are provided outside the tissue and/or living being. The process of influencing bioactivity transmits the control information signals.

Those skilled in the art will realize that this invention is capable of embodiments that are different from those shown and that details of the structure of the disclosed invention can be changed in various manners without departing from the scope of this invention. Accordingly, the drawings and descriptions are to be regarded as including such equivalents as do not depart from the spirit and scope of the invention.

### BRIEF DESCRIPTION OF THE DRAWINGS

Further features and advantages of the invention are best understood and will become apparent with reference to the accompanying drawings in which:

FIG. 1 depicts a diagrammatic illustration of a device for the detection of electrical bioactivity according to one embodiment of the invention;

FIG. 2 depicts details of the structure of the device of FIG. 1; and

FIG. 3 depicts a diagrammatic illustration of a device for influencing electrical bioactivity according to one embodiment of the invention.

### DETAILED DESCRIPTION

The invention is based on the finding that by providing a voltage-sensitive switch, an energy receiver, and transmitter or control information receiver as shown and described, it is possible to implement a miniaturization of the devices for detecting and/or influencing electrical bioactivity by coding information in the transmission properties, such as transmit amplitude and/or transmit frequency, of the transmitter and/or in properties of the control information signals and/or of the control information receiver, in analog fashion, such that the energy receiver and the transmitter and/or control information receiver can be operated in parallel in time. Moreover, separating the function of the energy receiver from the function of the transmitter and/or control information receiver enables unique identification of the devices among one another and/or separate driving of the devices when only one transmitter or control information receiver is provided per device, and unique identification of transmitters and/or separate driving of the control information receiver when more than one transmitter or more than one control information receiver is provided per implantable device.

Since a very small number of modules participate in the signal processing owing, inter alia, to the use of the transmission properties of the transmitter and/or properties of the control information signals (such as amplitude and/or frequency) and/or of the control information receiver for the purpose of coding the transmitted information in analog fashion, the devices according to the invention are extremely quick to react and thus enable the electrical bioactivity to be detected and/or influenced yet more closely in real time.

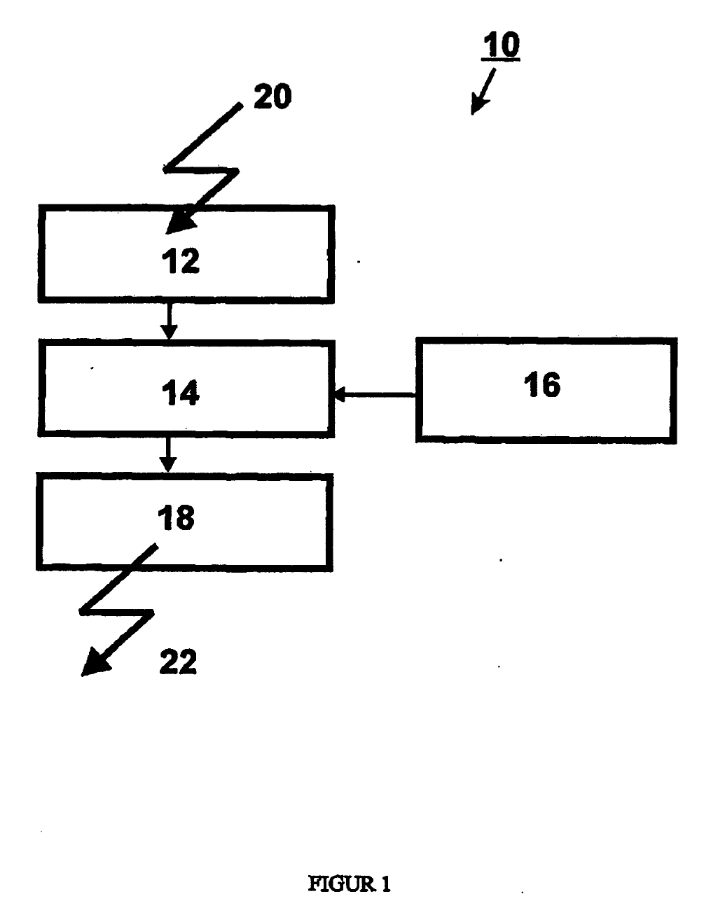

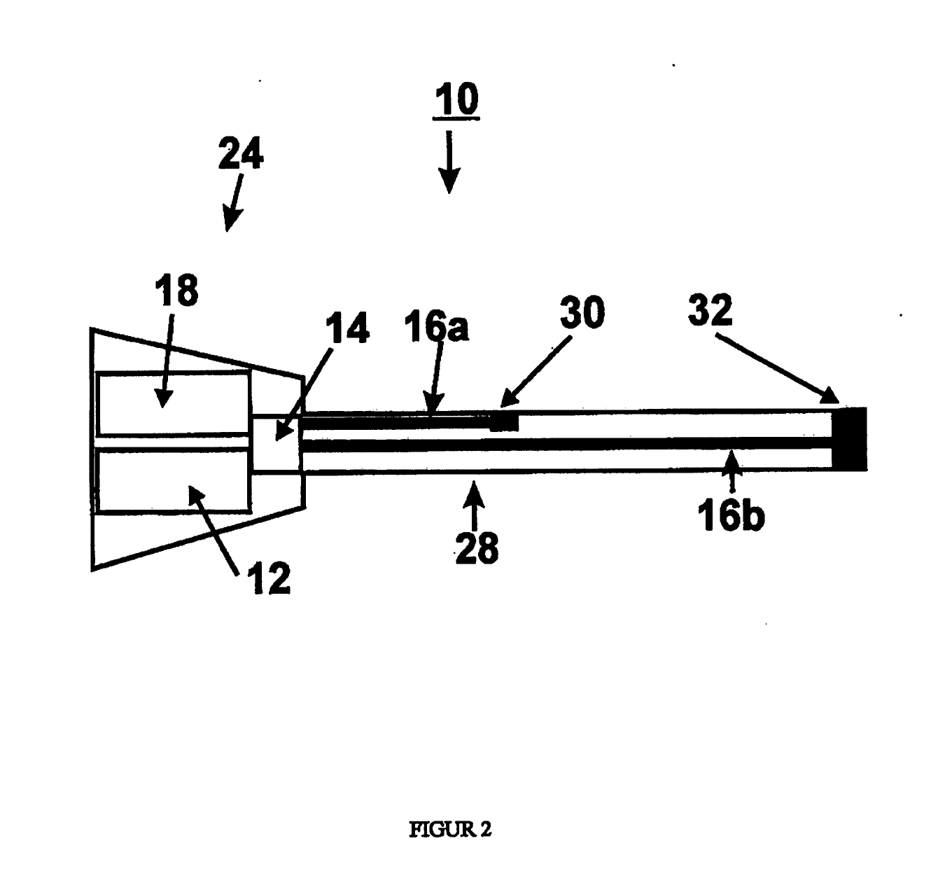

Referring now to FIGS. 1 and 2, a device 10 for implantation in a living being for detecting electrical bioactivity in accordance with a particular embodiment of the invention comprises an energy receiver 12, a voltage-sensitive switch 14, two measuring electrodes 16a and 16b which are generally referred to by reference numeral 16 in FIG. 1, and a transmitter 18. The energy receiver 12 receives electro-magnetic waves 20 from outside a tissue (not shown) and converts these into electrical energy. In the present example, the electrical energy is stored, for example, in one or more capacitor(s) (not shown) and is then passed on as required, for example, when the transmitter 18 is to transmit information. Alternatively, the electrical energy received by the energy receiver 12 can be passed on directly without intermediate storage at the transmitter 18 within the intended scope of the invention. It is further contemplated that in some embodiments, energy can be supplied by metabolism inherent to the body instead of via the energy receiver 12.

The voltage-sensitive switch 14 is arranged between the measuring electrode 16 and the transmitter 18. The voltage-sensitive switch 14 can be, for example, a voltage-sensitive resistor or a capacitor. The device 10 records the electrical bioactivity of, for example, nerve tissue (not shown) in the vicinity of the measuring electrode 16, and passes on this information to the transmitter 18. When the voltage difference in the nerve tissue reaches a specific voltage difference threshold value, the switch 14 switches on the transmitter 18. If a capacitor is employed as voltage-sensitive switch 14, the transmitter 18 is influenced by the switch 14 such that the change in voltage detected by the measuring electrode 16 in the surrounding nerve tissue can be gathered from the information transmission signal of the transmitter 18.

The switch 18 converts electric current from the energy receiver 12 into electromagnetic waves 22. The electromagnetic waves contain information relating to, for example, action potentials and/or changes in voltage differences that are detected by means of the measuring electrode 16, and therefore supply an information transmission signal. In FIGS. 1 and 2, the transmitter 18 comprises an open resonant circuit (not shown). When more than one such device and more than one transmitter are used, these can, for example, be configured so as to be distinguishable by different wavelengths and/or pulsed signals.

It is contemplated that in some embodiments, a number of energy receivers, voltage-sensitive switches and transmitters can also be present on a device in order to detect electrical bioactivity. This enables some embodiments to obtain information relating to the spatial distribution of the local bioactivity, e.g. tetrodes. The density of the devices may be limited substantially by the separability of the various information signals from the transmitter, with for example, different wavelengths, and by the ready size of the devices.

The device 10 can be fabricated as an integrated circuit (IC) and may incorporate nano/microsystem technology.

Referring now to FIG. 2, the device 10 comprises a head region 24, in which the energy receiver 12, the voltage-sensitive switch 14 and the transmitter 18 are located on a structure 26 resembling a printed circuit board, and a spur 28, which is thin and extends away from the head region 24. The spur 28 has two measuring electrodes 16a and 16b, each having a respective contact point 30 and 32. Except for these contact points 30 and 32, the complete device 10 is provided with an electrically insulating varnish (not shown). The varnish should exhibit properties that reduce stimulation of the surrounding tissue (not shown). In some embodiments, the device 10 can be equipped with barbs (not shown) to prevent the device 10 from slipping. Apart from the measuring electrodes 16a and 16b and contact points 30 and 32, the spur 28 requires no additional components.

A number of such devices 10 can be placed tightly next to one another and at variable spacings and yet be located in a fixed position in a tissue such as, for example, in the brain.

The device 10 can be used for real time detection of, for example, the activity of nerve cells, and for emitting a corresponding information signal from the transmitter 18.

When a number of such devices 10 are used, each device can use a frequency for the electromagnetic waves supplying energy, and a dedicated frequency, i.e. a dedicated channel, can be used for the electromagnetic waves emitted by the transmitter 18. Therefore, information can be transmitted to the outside by each device in an at least virtually continuous fashion without a pause in transmission and virtually without a reaction time.

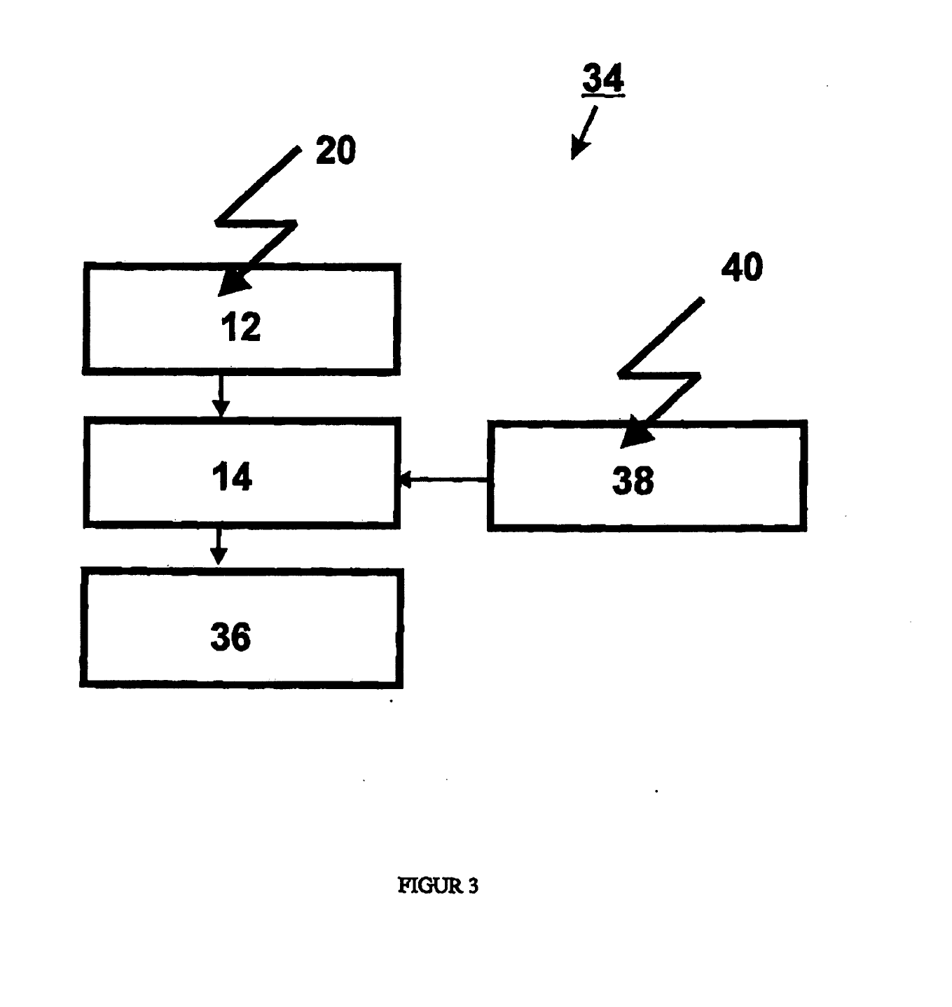

FIG. 3 depicts a device 34 shown for influencing electrical bioactivity comprising an energy receiver 12, a voltage-sensitive switch 14, two electrodes which are generally referred to by reference numeral 36, and a control information receiver 38.

Like the device shown and described in FIGS. 1 and 2, the energy receiver 12 of FIG. 3 receives electromagnetic waves 20 from outside and converts these into electrical energy, which can be stored in one or more capacitor(s) (not shown) and then discharged for influencing the electrical bioactivity of a tissue and/or living being. Alternatively, the electrical energy can be passed on directly to the electrodes 36 without intermediate storage. In some embodiments, the energy is supplied by the metabolism inherent in the body.

The control information receiver 38 receives control information in the form of electromagnetic waves 40 and converts these into electric current. This current is used to control the voltage-sensitive switch 14. When more than one device 34 or more than one control information receiver 38 is used, the control information receiver 38 can be configured to respond, for example, to only a very specific wavelength of the electromagnetic waves 40 which differs from other wavelengths being used.

In some embodiments, the voltage-sensitive switch 14 can be a resistor or a capacitor and be driven by a control signal from the control information receiver 38 in order to control a flow of current from the energy receiver 12 to the electrodes 36 in the tissue. This can involve converting the control signal into a resistance value. In such case, the control signal can be a function of the control information transmitted via the electromagnetic waves 40.

In some embodiments, a number of energy receivers 12, voltage-sensitive switches 14 and control information receivers 38 may be present in the case of a device 34 such that the local bioactivity can be influenced in three dimensions.

The density of the devices 34 is limited by the separability of the various control signals, the various control information receivers, and the size of the devices 34.

It will be appreciated that both the device for detecting electrical bioactivity and the device for influencing electrical bioactivity according to the invention include a wireless energy supply, wireless control signal transmission and small dimensions, enabling a high density of detection points and influencing points.

Those skilled in the art will recognize that the various features of this invention described above can be used in various combinations with other elements without departing from the scope of the invention. Thus, the appended claims are intended to be interpreted to cover such equivalents as do not depart from the spirit and scope of the invention.

1. A device for implantation in a living being for detecting electrical bioactivity comprising:
two measuring electrodes for detecting a voltage difference representing bioactivity of tissue of the living being;
a wireless transmitter for transmitting information outside the tissue, the information relating to the bioactivity as represented by the voltage difference detected by said two measuring electrodes;
a wireless energy receiver for receiving energy from outside the tissue to supply said transmitter with electrical energy, said transmitter and said energy receiver operating in parallel in time; and
a voltage sensitive switch connected between said two measuring electrodes and said transmitter, said voltage sensitive switch being positioned for switching said transmitter such that information relating to changes in electrical bioactivity can be coded in analog fashion in the form of a change of at least one transmission property of said transmitter, and information relating to the identity of said transmitter can be coded in analog fashion in the form of at least one transmission property of said transmitter.

2. A device according to claim 1, the at least one transmission property of said transmitter is at least one of a transmit amplitude and a transmit frequency.

3. A device according to claim 1, said switch being configured such that said switch switches said transmitter to an on condition or an off condition when the detected voltage difference overshoots or undershoots a voltage threshold value which can be fixed in advance.

4. A device according to claim 1, said transmitter comprising a closed resonant circuit.

5. A device according to claim 1, said transmitter comprising a photodiode.

6. A device according to claim 1, said transmitter comprising an LED.

7. A device according to claim 1, said transmitter comprising a quantum well structure.

8. A device according to claim 1, said transmitter comprising a quantum line structure.

9. A device according to claim 1, further comprising at least two transmitters that can be distinguished on the basis of different transmission properties.

10. A device for implantation in a living being for influencing electrical bioactivity comprising:
two electrodes for applying an electric voltage in tissue of the living being to influence bioactivity;
an energy receiver for receiving energy from outside the tissue to supply the two electrodes with electrical energy;
a control information receiver for receiving wireless control information signals from outside the tissue, said energy receiver and said control information receiver operating in parallel in time;
a voltage-sensitive switch connected between said control information receiver and said two electrodes, said voltage-sensitive switch being positioned for switching a flow of electric current from said energy receiver to said electrodes under the control of said control information receiver; and
the identity of said control information receiver and the magnitude of the influence on the electrical bioactivity being coded in analog fashion by at least one of the frequency and amplitude of the control information signals.

11. A device according to claim 10, said switch being driven by said control information receiver such that a voltage pulse is generated between said two electrodes.

12. A device according to claim 10, said control information receiver comprising a closed resonant circuit.

13. A device according to claim 10, said control information receiver comprising a photodiode.

14. A device according to claim 10 further comprising at least two control information receivers that can be addressed separately on the basis of different reception properties.

15. A device according to claim 10, said energy receiver comprising a closed resonant circuit.

16. A device according to claim 10, said energy receiver comprising a photodiode.

17. A device according to claim 10, said energy receiver comprising a piezocrystal.

18. A device according to claim 10, said voltage-sensitive switch comprising a voltage-sensitive resistor.

19. A device according to claim 10, said voltage-sensitive switch comprising a chain of open field effect transistors.

20. A device according to claim 10, said voltage-sensitive switch comprising an electrooptic switch.

21. A device according to claim 20, said electrooptic switch comprising an LED and a photodiode.

22. A device according to claim 10, said device further comprising an integrated circuit.

23. A device according to claim 10, said device being at least partially encapsulated within an electrically insulating material.

24. A device according to claim 10, at least one of said electrodes comprising a spur.

25. A device according to claim 10, at least one of said electrodes comprising a capacitor.

26. A device according to claim 10, at least two of said energy receiver, said two electrodes, said control information receiver, and said voltage sensitive switch, is implanted within the tissue.

27. A device according to claim 10, at least one of said energy receiver, said two electrodes, said control information receiver, and said voltage sensitive switch is positioned outside the tissue.
